(19) United States
(12) Patent Application Publication
(10) Pub. No.: US 2026/0324994 A1
(43) Pub. Date: Oct. 22, 2026

(54) PHOTOPLETHYSMOGRAPHY MEASUREMENT DEVICE

(71) Applicant: Quanta Computer Inc., Taoyuan City (TW)

(72) Inventors: Yung-Ming CHUNG, Taoyuan City (TW); Jia-Li SUNG, Taoyuan City (TW)

(21) Appl. No.: 19/227,845

(22) Filed: Jun. 4, 2025

(30) Foreign Application Priority Data
Apr. 22, 2025 (TW) 114115153

(51) Int. Cl. A61B 5/024

(52) U.S. Cl. CPC A61B 5/02427; A61B 5/02416; A61B 2562/0233

(57) ABSTRACT

A photoplethysmography measurement device is provided, including a light source, a light sensor, a signal converter, and a processor. The light source generates an emitted signal. The light sensor receives a reflection signal generated by a target object reflecting the emitted signal. The light sensor generates an analog signal corresponding to the reflection signal. The signal converter converts the analog signal into a digital signal, and the digital signal has an amplitude. The processor enters a measurement mode when the amplitude is greater than or equal to a predetermined amplitude for a duration that is greater than or equal to a preset time.

### CROSS REFERENCE TO RELATED APPLICATIONS

This application claims priority of Taiwan Patent Application No. 114115153, filed on Apr. 22, 2025, the entirety of which is incorporated by reference herein.

### BACKGROUND OF THE INVENTION

### Field of the Invention

The present invention relates to a photoplethysmography measurement device and, in particular, it relates to a photoplethysmography measurement device that can perform measurements stably and accurately.

### Description of the Related Art

Photoplethysmography (PPG) is a method for measuring the volume of a target object using optical means, typically used to measure signals related to the physiological state of a subject. However, each measurement may be subject to errors due to various factors. In order to avoid these errors from having an unpredictable impact on the measurement results, or to prevent abnormal measurement results (such as noise) from being considered during the measurement process and affecting the judgment, a more precise solution that can filter the measurement results is needed.

### BRIEF SUMMARY OF THE INVENTION

An embodiment of the present invention provides a photoplethysmography measurement device, including a light source, a light sensor, a signal converter, and a processor. The light source generates an emitted signal. The light sensor receives a reflection signal generated by a target object reflecting the emitted signal. The light sensor generates an analog signal corresponding to the reflection signal. The signal converter converts the analog signal into a digital signal, and the digital signal has an amplitude. The processor enters a measurement mode when the amplitude is greater than or equal to a predetermined amplitude for a duration that is greater than or equal to a preset time.

### BRIEF DESCRIPTION OF THE DRAWINGS

The present invention can be more fully understood by reading the subsequent detailed description and examples with references made to the accompanying drawings, wherein:

FIG. 1 shows a schematic diagram of a photoplethysmography (PPG) measurement device, as demonstrated according to the embodiment disclosed herein;

FIG. 2 shows a timing diagram illustrating the measurement of the target object by the PPG measurement device under different measurement conditions;

FIG. 3 shows a flowchart illustrating the operation of the PPG measurement device; and

FIG. 4A to 4C show schematic diagrams illustrating the operation of the PPG measurement device.

### DETAILED DESCRIPTION OF THE INVENTION

The following description outlines multiple embodiments with reference to the accompanying drawings. In these drawings, similar reference numerals are used to represent similar or identical components. The drawings are not necessarily drawn to scale and are for illustrative purposes only, intended to show the aspects and features of the present disclosure. A substantial amount of specific details, relationships, and methods are explained to provide a complete understanding of the specific aspects and features of the disclosure. However, it will be understood by those skilled in the relevant art that these aspects and features may be practiced without one or more of the specific details, using alternative relationships or methods. In certain instances, well-known structures or operations are not detailed for clarity. The various embodiments disclosed are not necessarily limited to the order of actions or events as illustrated, and some actions may occur in a different sequence and/or concurrently with other actions or events. Furthermore, not all actions depicted in the illustrations are required to implement the specific aspects and features of the present disclosure.

Photoplethysmography (PPG) is a method that can be used for non-invasive blood pressure and/or blood oxygen measurement. When a pulse is detected, the reflected light changes according to the volume changes in the blood vessels, thus forming a PPG signal with varying values. General blood pressure and/or blood oxygen PPG measurement devices (e.g., wearable devices) typically perform measurements by pressing against the skin (e.g., on the back of the wrist or fingertip). However, such measurement methods may suffer from reduced reliability and stability of the results if they are not at the optimal measurement angle (e.g., closely fitting the skin) or if the measurement duration is insufficient. The device and method disclosed herein provide a solution that filters out stable measurement results, thereby reducing measurement errors and increasing data reliability.

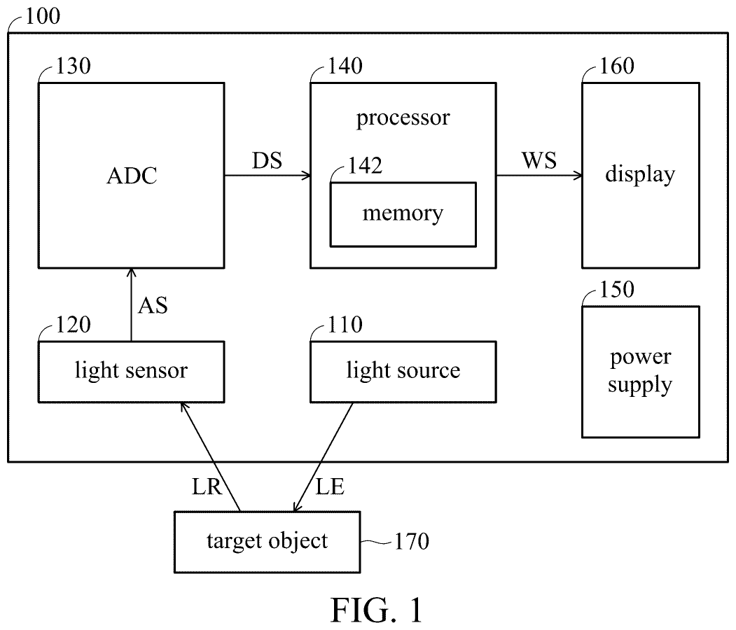

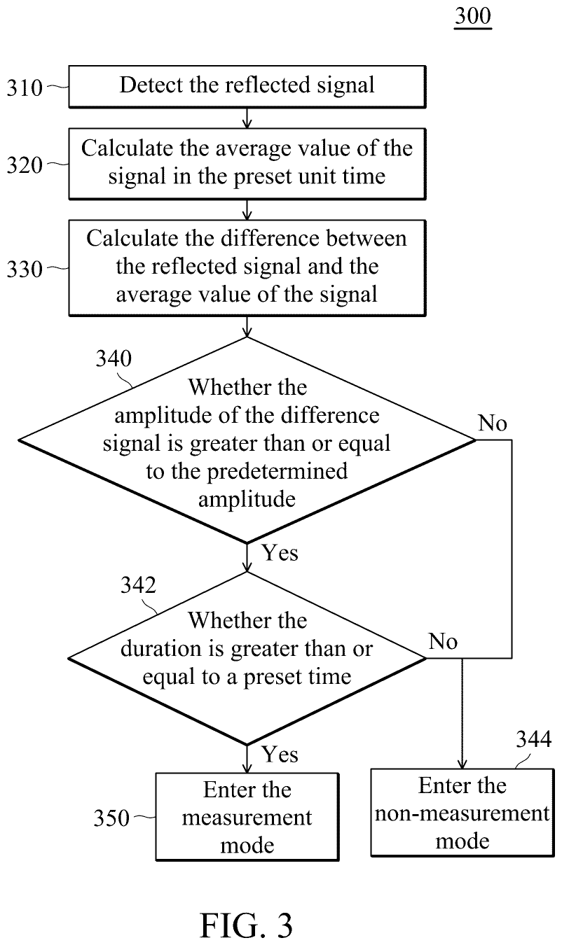

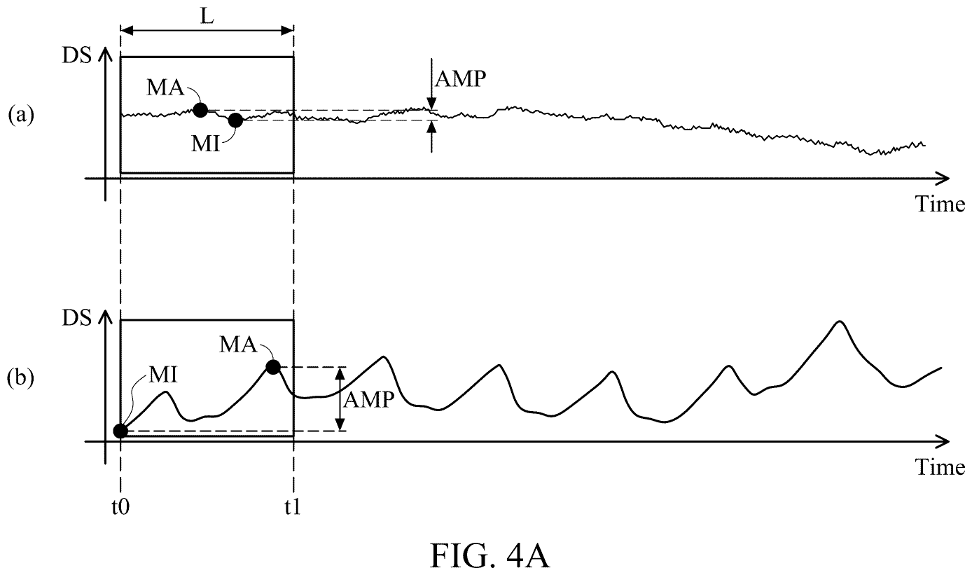

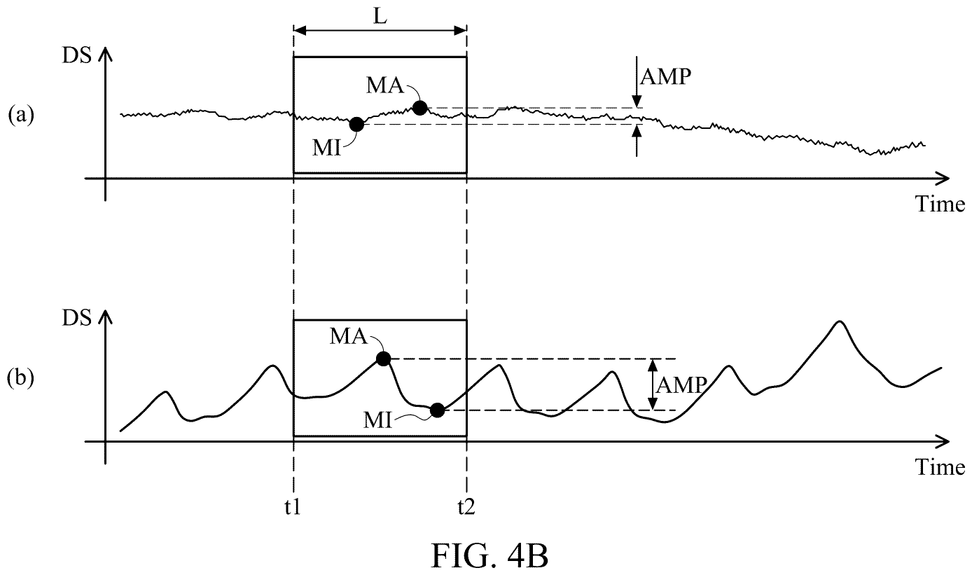

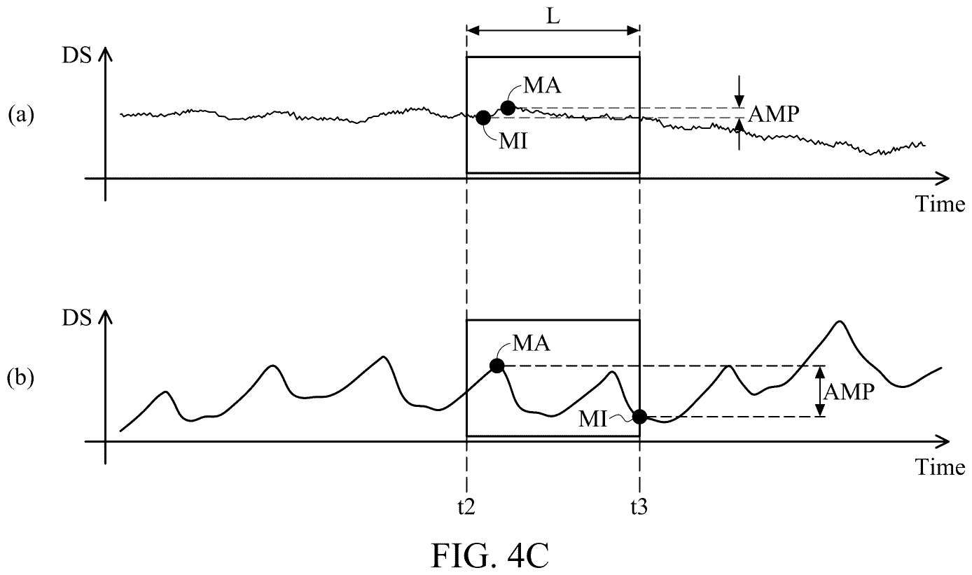

FIG. 1 shows a schematic diagram illustrating the measurement of the photoplethysmography (PPG) measurement device 100 according to the embodiment disclosed herein. The PPG measurement device 100 includes a light source 110, a light sensor 120, an analog-to-digital converter (ADC) 130, and a processor 140. The light source 110 generates an emitted signal LE and irradiates a target object 170 (e.g., skin or blood vessels beneath the skin) to produce a reflected signal LR. The light sensor 120 receives the reflected signal LR and converts it into an analog signal AS, which is output to the ADC 130 (or any suitable signal converter). The ADC 130 converts the analog signal AS into a digital signal DS and outputs the digital signal DS to the processor 140 for subsequent processing as shown in FIG. 3 and FIGS. 4A to 4C.

The PPG measurement device 100 may optionally include a display 160 and a power supply 150. After detecting and/or measuring the digital signal DS, the processor 140 may further convert the digital signal DS into a display signal WS and output it to the display 160 (e.g., a screen of a wearable device) to present the waveform and values corresponding to the digital signal DS (e.g., the measured pulse/oxygen saturation value and the pulse waveform diagram). Additionally, the PPG measurement device 100 is powered by an internal power supply 150 (e.g., a battery). Therefore, to reduce power consumption during measurement and extend operating time, while also increasing the stability and reliability of the measured data, the PPG measurement device 100 may operate in a non-measurement mode and a measurement mode. When an appropriate or meaningful reflected signal LR (and the corresponding digital signal DS) is detected in the non-measurement mode, the PPG measurement device 100 then switches to the measurement mode to measure and actually obtain the optical volume change data of the target object 170.

For example, after the PPG measurement device 100 is powered on (i.e., powered by the internal power supply 150 of the PPG measurement device 100), the PPG measurement device 100 (or the processor 140) enters a non-measurement mode. The light source 110 generates an emitted signal LE, which is reflected by the target object 170 to the light sensor 120. The reflected signal LR is then converted into a digital signal DS by the light sensor 120 and the ADC 130. Next, the processor 140 measures the amplitude AMP of the digital signal DS and compares the amplitude AMP with a predetermined amplitude. When the amplitude AMP is greater than or equal to the predetermined amplitude, the PPG measurement device 100 enters the measurement mode. The processor 140 then captures the amplitude variations of the digital signal DS to generate data corresponding to the optical volume changes of the target object 170. If the amplitude AMP of the digital signal DS is less than the predetermined amplitude, the PPG measurement device 100 remains in the non-measurement mode and continues comparing the amplitude AMP with the predetermined amplitude.

In one embodiment, the emitted signal LE generated by the light source 110 may be of different colors of light, such as red light, infrared light, or green light, allowing the PPG measurement device 100 to switch between them when measuring different target data. However, the PPG measurement device 100 may also include multiple light sources 110, with each light source 110 emitting a single color of light. Correspondingly, the light sensor 120 may be capable of receiving and converting multiple types of reflected signals LR, or the PPG measurement device 100 may include multiple light sensor 120, with each light sensor 120 dedicated to receiving a specific color of light.

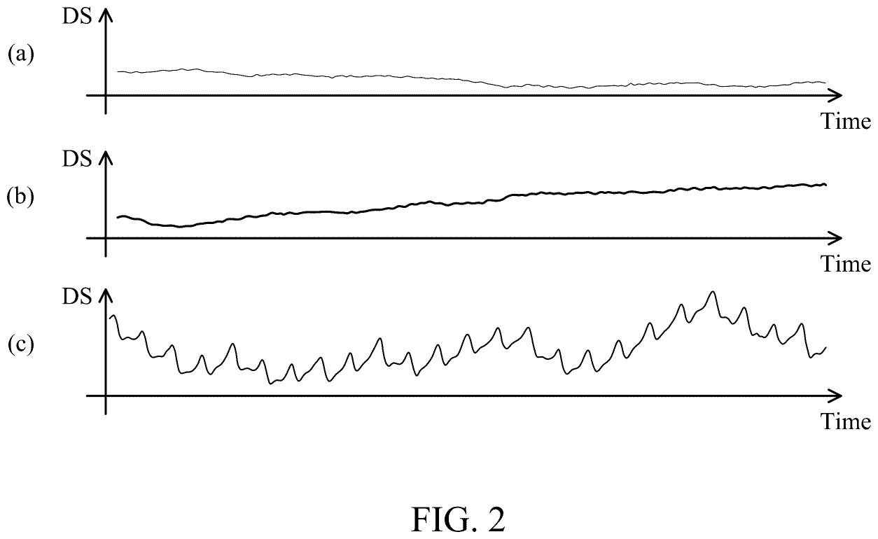

FIG. 2 is a timing diagram showing the measurement of the target object 170 by the PPG measurement device 100 under different measurement conditions. Under measurement condition (a), the timing diagram of the digital signal DS does not show significant amplitude variation, which suggests that the PPG measurement device 100 may not be properly aligned (e.g., parallel to or in contact with) the target object 170, or that the target object 170 does not exhibit periodic optical volume changes. Therefore, when the processor 140 performs amplitude comparison, it determines not to enter the measurement mode. Under measurement condition (b), although the digital signal DS shows more noticeable level variation compared to condition (a), there is still no significant amplitude variation. Therefore, the processor 140 likewise determines not to enter the measurement mode. Under measurement condition (c), the digital signal DS exhibits significant amplitude variation. As a result, when the processor 140 performs the amplitude comparison operation, it may determine to enter the measurement mode. In other words, only under measurement condition (c) is it determined that the digital signal DS indicates proper usage of the PPG measurement device 100 by the user.

FIG. 3 is a flowchart 300 illustrating the operation of the PPG measurement device 100. After the PPG measurement device 100 is powered on, the light source 110 generates an emitted signal LE and projects it onto the target object 170. Next, in step 310 of the flowchart 300, the light sensor 120 receives and detects the reflected signal LR, which is generated by the target object 170 reflecting the emitted signal LE. The light sensor 120 converts the reflected signal LR into an analog signal AS and outputs it to the ADC 130. The ADC 130 then converts the analog signal AS into a digital signal DS and outputs it to the processor 140. Then, in steps 320 and 330, the processor 140 calculates the amplitude AMP of the digital signal DS.

Since the actual voltage of a PPG signal is typically quite small and not easily used for direct amplitude determination, the amplitude AMP of the digital signal DS is defined in units of “DC values.” The voltage corresponding to one DC value can be converted as follows:

$$V = \frac{N}{{2}^{A}} \times B \qquad Equation ( 1 )$$

Here, V represents the voltage of the analog signal AS, N is the DC value of the digital signal DS, A is the resolution (e.g., number of bits) of the ADC 130 used to convert the analog signal AS into the digital signal DS, and B is the maximum voltage supplied by the power supply 150. Assuming a resolution of 19 bits, a maximum supplied voltage of 2V, and a DC value of 108000 for the digital signal DS, the corresponding voltage of the analog signal AS can be calculated using Equation (1) to be 0.41V. By using a digital signal DS with higher resolution, the PPG measurement device 100 can more accurately estimate the amplitude AMP.

However, as the chip used in the PPG measurement device 100 changes (e.g., if the maximum voltage or resolution changes), the DC value of the digital signal DS will also change. Therefore, it is necessary to standardize the values of the digital signal DS. In step 320, the processor 140 obtains the average value of the digital signal DS over a preset unit time L (e.g., the average DC value). Then, in step 330, the processor 140 subtracts this average value from the digital signal DS to obtain a difference signal. The waveform of the difference signal is the same as or similar to the digital signal DS, but with a DC value close to zero at the origin. At this point, the amplitude AMP of the digital signal DS can be derived by determining the maximum and minimum values of the difference signal within the preset unit time L.

After deriving the amplitude AMP, the process moves to step 340, where the processor 140 compares the amplitude AMP (the maximum and minimum values of the difference signal within the preset unit time L) with a predetermined amplitude. For example, referring to FIG. 2, when the target object 170 is absent, when it does not exhibit periodic volume changes, or when it is not aligned with the PPG measurement device 100 (i.e., in measurement states (a) and (b)), the amplitude AMP of the digital signal DS is not significant (i.e., the maximum and minimum values of the difference signal within the preset unit time L are less than the predetermined amplitude). When the target object 170 is properly aligned with (e.g., parallel to or in contact with) the PPG measurement device 100 (i.e., measurement state (c)), the digital signal DS exhibits a significant amplitude AMP (i.e., the maximum and minimum values of the difference signal within the preset unit time L are greater than or equal to the predetermined amplitude). Therefore, by appropriately setting this predetermined amplitude, valid measurement data can be effectively filtered. Additionally, it should be noted that since the unit of the amplitude AMP is in DC values, the unit of the predetermined amplitude is also in DC values. In other words, although the numerical value of the predetermined amplitude may vary depending on the chip used in the PPG measurement device 100, the actual voltage corresponding to the predetermined amplitude remains unchanged.

If the determination in step 340 is affirmative, the process proceeds to step 342, where the processor 140 determines whether the duration during which the amplitude AMP is greater than or equal to the predetermined amplitude is greater than or equal to a preset time. This is because, during the operation of the PPG measurement device 100, sudden movements or interference from the surrounding environment may cause pulse peaks (e.g., the digital signal DS may momentarily exhibit a very high DC value). At this point, in order to prevent the processor 140 from mistakenly determining that the amplitude AMP is greater than (or equal to) the predetermined amplitude due to a pulse peak, a duration condition is added in step 342 to ensure data accuracy (as explained in more detail below). If the determination in step 342 is affirmative, the process proceeds to step 350, where the PPG measurement device 100 (or the processor 140) enters the measurement mode and begins capturing data from the digital signal DS or using the previously evaluated digital signal DS data to calculate target parameters (e.g., pulse rate, blood oxygen level, etc.).

If the determination in step 342 is negative, the process proceeds to step 344, where the PPG measurement device 100 (or the processor 140) enters the non-measurement mode. The flowchart 300 then returns to step 310. Similarly, if the determination in step 340 is negative, the process also proceeds to step 344, and the PPG measurement device 100 (or the processor 140) enters the non-measurement mode. The flowchart 300 then returns to step 310.

FIGS. 4A to 4C are schematic diagrams illustrating the operation of the PPG measurement device 100. In FIGS. 4A to 4C, measurement condition (a) represents abnormal operation, for example, the target object 170 may be absent, may not have characteristics that generate a PPG signal, or may not be properly aligned with the PPG measurement device 100. Measurement condition (b) represents normal operation, for instance, the target object 170 has characteristics capable of generating a PPG signal and is properly aligned with the PPG measurement device 100. In step 342, the preset time can be a specific multiple of the preset unit time L. In the following explanation, it is assumed that the preset time is 3L (i.e., three preset unit times L).

Between time t0 and t1 (considered as a first interval with a duration that is equal to the preset unit time L), the digital signal DS generated under measurement condition (a) has a maximum value MA and a minimum value MI, and the digital signal DS generated under measurement condition (b) also has a maximum value MA and a minimum value MI. However, as shown in FIG. 4A, the amplitude AMP under measurement condition (a) is smaller, while the amplitude AMP under condition (b) is larger. Therefore, by comparing the amplitude AMP obtained under both measurement conditions with an appropriate predetermined amplitude, the PPG measurement device 100 can be kept in the non-measurement mode under condition (a) (since it is less than the predetermined amplitude). Consequently, the duration during which the amplitude AMP is greater than or equal to the predetermined amplitude is zero.

However, under measurement condition (b), the duration during which the amplitude AMP is greater than or equal to the predetermined amplitude is only one preset unit time L, which does not meet the requirement that the duration be greater than or equal to the preset time (assumed here to be 3L). Therefore, the PPG measurement device 100 will not enter the measurement mode, but the duration for which the amplitude AMP is greater than or equal to the predetermined amplitude is recorded as L (i.e., the duration of the first interval).

Next, refer to FIG. 4B. Between time t1 and t2 (considered as a second interval with a duration that is equal to the preset unit time L), the digital signal DS under both measurement conditions (a) and (b) has respective maximum values MA and minimum values MI. However, as shown in the figure, measurement condition (a) results in a smaller amplitude AMP, while measurement condition (b) results in a larger amplitude AMP. Therefore, by comparing the amplitudes with the predetermined amplitude, it can be concluded that under measurement condition (a), the amplitude AMP is less than the predetermined amplitude, whereas under condition (b), the amplitude AMP is greater than or equal to the predetermined amplitude.

Therefore, under measurement condition (a), the PPG measurement device 100 will not enter the measurement mode, and the duration during which the amplitude AMP is greater than or equal to the predetermined amplitude remains zero. Under measurement condition (b), since the amplitude AMP is greater than or equal to the predetermined amplitude, the processor 140 subsequently determines the duration for which the amplitude AMP remains greater than or equal to the predetermined amplitude. As of time t2, the duration during which the amplitude AMP is greater than or equal to the predetermined amplitude is only 2L (i.e., two preset unit times L), which does not meet the requirement of the duration being greater than or equal to the preset time (assumed here to be 3L). Therefore, the PPG measurement device 100 will not enter the measurement mode, but the duration during which the amplitude AMP is greater than or equal to the predetermined amplitude is recorded as 2L (i.e., the total duration of the first and second intervals).

Next, refer to FIG. 4C. Between time t2 and t3 (considered as a third interval with a duration that is equal to the preset unit time L), the digital signal DS under both measurement conditions (a) and (b) has respective maximum values MA and minimum values MI. However, as shown in the figure, measurement condition (a) results in a smaller amplitude AMP, while measurement condition (b) results in a larger amplitude AMP. Therefore, by comparing the amplitudes with the predetermined amplitude, it can be concluded that under measurement condition (a), the amplitude AMP is less than the predetermined amplitude, while under condition (b), the amplitude AMP is greater than or equal to the predetermined amplitude.

Therefore, under measurement condition (a), the PPG measurement device 100 will not enter the measurement mode, and the duration during which the amplitude AMP is greater than or equal to the predetermined amplitude remains zero. Under measurement condition (b), since the amplitude AMP is greater than or equal to the predetermined amplitude, the processor 140 subsequently determines the duration for which the amplitude AMP remains greater than or equal to the predetermined amplitude. As of time t3, the duration during which the amplitude AMP is greater than or equal to the predetermined amplitude reaches 3L (i.e., three preset unit times L), which meets the requirement of the duration being greater than or equal to the preset time (assumed here to be 3L). Therefore, the PPG measurement device 100 enters the measurement mode, and the processor 140 acquires the digital signal DS to derive the target data (e.g., pulse rate, blood oxygen level, etc.).

It should be noted that when the PPG measurement device 100 enters the measurement state, this means that during the preset time intervals (such as the first, second, and third intervals mentioned earlier), the amplitude AMP of the digital signal DS also meets the amplitude condition required in the measurement state. Therefore, if the PPG measurement device 100 successfully enters the measurement state, the digital signal DS measured during the preset time can also be considered part of the measurement data. As a result, the PPG measurement device 100 further includes a memory 142 to record the digital signal DS when the amplitude AMP is greater than or equal to the predetermined amplitude during the non-measurement state.

The following will explain the predetermined amplitude disclosed herein through an example. The table below presents an example of the digital signal DS and the amplitude AMP voltage measured for different target objects 170 and measurement conditions, where the maximum supply voltage is assumed to be 2V and the resolution is 19 bits:

TABLE 1

| | digital signal | |
|---|---|---|
| Measurement conditions | DS(V) | amplitude AMP(V) |
| Lay flat on table | 0.001-0.07 | <0.0004 (First range) |
| At an angle to the table | 0.1-0.8 | <0.0007 (Second range) |
| Measure wrist or fingertips | 0.38-1.14 | 0.001-0.08 (Third range) |

As shown in Table 1 above, when the target object 170 does not exist or is not properly aligned with the PPG measurement device 100 (e.g., lying flat on a table or at an angle to the table), the voltage of the digital signal DS is smaller. However, when the target object 170 is present (e.g., measuring the wrist or fingertip) and is parallel to or in contact with the PPG measurement device 100, the voltage of the digital signal DS is larger.

However, according to Table 1, if only the digital signal DS is measured, it is possible to achieve a voltage that corresponds to a normal measurement operation, even if the correct measurement procedure was not followed. For example, when the PPG measurement device 100 is at an angle to the table, the voltage corresponding to the digital signal DS could be 0.8V, which exceeds the possible minimum voltage of 0.38V when measuring the wrist or fingertip. Therefore, the PPG measurement device 100 further limits the amplitude AMP of the digital signal DS, categorizing the amplitude AMP into three ranges: the first range corresponding to when the device is placed flat on a table, the second range corresponding to when the device is at an angle to the table, and the third range corresponding to when measuring the wrist or fingertip. This allows for effective determination of whether the device has entered the measurement state. For example, when the device is placed flat on a table, the amplitude AMP is less than 0.0004 V. When there is an angle between the device and the table, the amplitude AMP is less than 0.0007 V. However, when measuring the wrist or fingertip, the amplitude AMP can range from 0.001 V to 0.08 V. In other words, under different measurement conditions, the amplitude AMP shows more distinct variations compared to the actual voltage corresponding to the digital signal DS. Therefore, when the target object 170 is present and aligned with or in contact with the PPG measurement device 100, the lower limit of the third amplitude range associated with valid measurements is used as the predetermined amplitude.

Finally, the PPG measurement device 100 can also impose constraints on the actual voltage corresponding to the digital signal DS. For example, as shown in Table 1, the actual voltage corresponding to the digital signal DS when the device is placed flat on a table ranges from 0.001 V to 0.07 V, while the actual voltage obtained from measuring the wrist or fingertip ranges from 0.38 V to 1.14 V. Therefore, by selecting an appropriate voltage threshold within the 0.07 V to 0.38 V range (e.g., requiring the actual voltage corresponding to the digital signal DS to be greater than or equal to 0.22 V), it is also possible to filter out certain data that do not meet the criteria for proper measurement conditions, thereby supporting data filtering and enhancing the stability and reliability of the measurement data.

The present disclosure provides a PPG measurement device that filters PPG signals reflected from a target object to deliver more stable and reliable measurement data. For example, to reduce power consumption and conserve battery life when measurement is not required, the disclosure presents a determination method: the device enters the measurement state and begins acquiring the detected PPG signal only when the amplitude of the measured PPG signal is greater than or equal to a predetermined amplitude, and the duration of this condition is greater than or equal to a preset time. In addition, the present disclosure also provides an auxiliary determination condition: the PPG measurement device enters the measurement state and begins acquiring the detected PPG signal only when the actual voltage corresponding to the measured PPG signal is greater than or equal to a certain threshold. After acquiring the PPG signal, the device can generate a display signal based on the photoplethysmographic characteristics or light absorption spectrum of the target object carried by the PPG signal. This display signal can then be presented via a display to show either a pulse waveform diagram corresponding to the photoplethysmographic characteristics (e.g., a pulse waveform chart), or numerical values corresponding to the light absorption spectrum (e.g., blood oxygen levels), for the user of the PPG measurement device to view.

What is claimed is:

1. A photoplethysmography (PPG) measurement device, including:
a light source, configured to generate an emitted signal;
a light sensor, configured to receive a reflected signal generated by a target object reflecting the emitted signal, and to generate an analog signal corresponding to the reflected signal;
a signal converter, configured to convert the analog signal into a digital signal, wherein the digital signal has an amplitude; and
a processor, configured to enter a measurement mode when a duration during which the amplitude is greater than or equal to a predetermined amplitude is greater than or equal to a preset time.

2. The photoplethysmography (PPG) measurement device as claimed in claim 1, wherein the operation of acquiring the digital signal when the duration during which the amplitude is greater than or equal to the predetermined amplitude is greater than or equal to the preset time includes:
the processor measuring the amplitude of the digital signal in a first interval;
in response to the amplitude in the first interval being greater than or equal to the predetermined amplitude, the processor measuring the amplitude of the digital signal in a second interval;
in response to the amplitude in the second interval being greater than or equal to the predetermined amplitude, the processor measuring the amplitude of the digital signal in a third interval; and
in response to the amplitude in the third interval being greater than or equal to the predetermined amplitude, the processor entering the measurement mode,
wherein the preset time is the total duration of the first interval, the second interval, and the third interval, and the first interval, the second interval, and the third interval are sequentially connected.

3. The photoplethysmography (PPG) measurement device as claimed in claim 2, wherein in response to the processor entering the measurement mode, the processor further acquires portions of the digital signal that fall within the first interval, the second interval, or the third interval.

4. The photoplethysmography (PPG) measurement device as claimed in claim 2, wherein in response to the amplitude in the first interval, the second interval, or the third interval being less than the predetermined amplitude, the processor enters a non-measurement mode.

5. The photoplethysmography (PPG) measurement device as claimed in claim 1, wherein the preset time includes a plurality of intervals, and the amplitude of the digital signal is defined, for each of the intervals, as a difference between a maximum voltage value and a minimum voltage value of the digital signal.

6. The photoplethysmography (PPG) measurement device as claimed in claim 1, wherein:
when the target object does not exist, or the light sensor does not receive the reflected signal, the amplitude is within a first range;
when there is an angle of inclination between the light sensor and the target object, the amplitude is within a second range; and
when the light sensor is parallel to or in contact with the target object, the amplitude is within a third range,
wherein the third range is greater than the second range, the second range is greater than the first range, and the predetermined amplitude is a lower limit of the third range.

7. The photoplethysmography (PPG) measurement device as claimed in claim 1, further comprising a display configured to receive a display signal corresponding to the digital signal from the processor.

8. The photoplethysmography (PPG) measurement device as claimed in claim 7, wherein the display signal corresponds to a pulse waveform representing photoplethysmographic characteristics of the target object, or a numerical value representing light absorption spectrum characteristics of the target object.

9. The photoplethysmography (PPG) measurement device as claimed in claim 1, wherein the reflected signal is a photoplethysmographic signal.

10. The photoplethysmography (PPG) measurement device as claimed in claim 1, wherein the emitted signal is red light, infrared light, or green light.
